(19) United States
(12) Patent Application Publication
(10) Pub. No.: US 2022/0272985 A1
(43) Pub. Date: Sep. 1, 2022

(54) Novel Stenotrophomonas Strains and Related Methods

(71) Applicants: Agriculture Victoria Services PTY LTD, Bundoora, Victoria (AU); Dairy Australia Limited, Southbank, Victoria (AU); Geoffrey Gardiner Dairy Foundation Limited, Melbourne, Victoria (AU)

(72) Inventors: Tongda Li, Southbank (AU); Ian Ross Tannenbaum, Bundoora (AU); Jatinder Kaur, Taylors Hill (AU); Christian Krill, Reservoir (AU); Timothy Ivor Sawbridge, Coburg (AU); Ross Mann, Coburg (AU); German Carlos Spangenberg, Bundoora (AU)

(73) Assignees: Agriculture Victoria Services PTY LTD, Bundoora, Victoria (AU); Dairy Australia Limited, Southbank, Victoria (AU); Geoffrey Gardiner Dairy Foundation Limited, Melbourne, Victoria (AU)

(21) Appl. No.: 17/627,793

(22) PCT Filed: Jul. 17, 2020
(86) PCT No.: PCT/AU2020/050737

§ 371 Date: Jan. 17, 2022

(30) Foreign Application Priority Data
Jul. 19, 2019 (AU) 2019902561

(51) Int. Cl. A01N 63/20; A01H 17/00; A01H 3/00; C05F 11/08; C12N 1/20

(52) U.S. Cl. CPC A01N 63/20; A01H 17/00; A01H 3/00; C05F 11/08; C12N 1/205; C12R 2001/01

(57) ABSTRACT

The present invention relates to an endophyte strain isolated from a plant of the Poaceae family, wherein said endophyte is a strain of Stenoptrophomonas rhizophila which provides bioprotection and/or biofertilizer phenotypes to plants into which it is inoculated. The present invention also discloses plants infected with the endophyte and related methods.

### FIELD OF THE INVENTION

The present invention relates to novel plant microbiome strains, plants infected with such strains and related methods.

### BACKGROUND OF THE INVENTION

Microbes represent an invaluable source of novel genes and compounds that have the potential to be utilised in a range of industrial sectors. Scientific literature gives numerous accounts of microbes being the primary source of antibiotics, immune-suppressants, anticancer agents and cholesterol-lowering drugs, in addition to their use in environmental decontamination and in the production of food and cosmetics.

A relatively unexplored group of microbes known as endophytes, which reside e.g. in the tissues of living plants, offer a particularly diverse source of novel compounds and genes that may provide important benefits to society, and in particular, agriculture.

Endophytes may be fungal or bacterial. Endophytes often form mutualistic relationships with their hosts, with the endophyte conferring increased fitness to the host, often through the production of defence compounds. At the same time, the host plant offers the benefits of a protected environment and nutriment to the endophyte.

Important forage grasses perennial ryegrass (Lolium perenne) are commonly found in association with fungal and bacterial endophytes. However, there remains a general lack of information and knowledge of the endophytes of these grasses as well as of methods for the identification and characterisation of novel endophytes and their deployment in plant improvement programs.

Knowledge of the endophytes of perennial ryegrass may allow certain beneficial traits to be exploited in enhanced pastures, or lead to other agricultural advances, e.g. to the benefit of sustainable agriculture and the environment.

There exists a need to overcome, or at least alleviate, one or more of the difficulties or deficiencies associated with the prior art.

### SUMMARY OF THE INVENTION

In one aspect, the present invention provides a substantially purified or isolated endophyte strain isolated from a plant of the Poaceae family, wherein said endophyte is a strain of Stenotrophomonas rhizophila which provides bioprotection and/or biofertilizer phenotypes to plants into which it is inoculated. In a preferred embodiment, the Stenotrophomonas rhizophila strain may be strain JB as described herein and as deposited with The National Measurement Institute of 1/153 Bertie Street, Port Melbourne, VIC 3207, Australia on 17^{th} May 2019 with accession number V19/009906.

As used herein the term “endophyte” is meant a bacterial or fungal strain that is closely associated with a plant. By “associated with” in this context is meant that the bacteria or fungus lives on, in or in close proximity to a plant. For example, it may be endophytic, for example living within the internal tissues of a plant, or epiphytic, for example growing externally on a plant.

As used herein the term “substantially purified” is meant that an endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

As used herein the term ‘isolated’ means that an endophyte is removed from its original environment (e.g. the natural environment if it is naturally occurring). For example, a naturally occurring endophyte present in a living plant is not isolated, but the same endophyte separated from some or all of the coexisting materials in the natural system, is isolated.

As used herein the term “bioprotection and/or biofertilizer” means that the endophyte possesses genetic and/or metabolic characteristics that result in a beneficial phenotype in a plantharbouring, or otherwise associated with, the endophyte. Such beneficial properties include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, relative to an organism not harboring the endophyte or harboring a control endophyte such as standard toxic (ST) endophyte.

The pests and/or diseases may include, but are not limited to, fungal and/or bacterial pathogens, preferably fungal. In a particularly preferred embodiment, the endophyte may result in the production of the bioprotectant compound in the plant with which it is associated.

As used herein, the term ‘bioprotectant compound’ is meant as a compound that provides or aids bioprotection to the plant with which it is associated against pests and/or diseases, such as bacterial and/or fungal pathogens. A bioprotectant compound may also be known as a ‘biocidal compound’.

In a particularly preferred embodiment, the endophyte produces a bioprotectant compound and provides bioprotection to the plant against bacterial and/or fungal pathogens. The terms bioprotectant, bioprotective and bioprotection (or any other variations) may be used interchangeably herein.

Thus, in a preferred embodiment, the present invention provides a method of providing bioprotection to a plant against bacterial and/or fungal pathogens, said method including infecting the plant with an endophyte as hereinbefore described and cultivating the plant.

In a particularly preferred embodiment the bioprotectant compound is spermidine or derivative, isomer and/or salt thereof.

The endophyte may be suitable as a biofertilizer to improve the availability of nutrients to the plant with which the endophyte is associated, including but not limited to improved tolerance to nutrient stress.

Thus, in a preferred embodiment, the present invention provides a method of providing biofertilizer to a plant, said method including infecting the plant with an endophyte as hereinbefore described and cultivating the plant.

The nutrient stress may be lack of or low amounts of a nutrient such as phosphate and/or nitrogen. The endophyte may be capable of growing in conditions such as low nitrogen and/or low phosphate and enable these nutrients to be available to the plant with which the endophyte is associated.

The endophyte may result in the production of organic acids and/or the solubilisation of phosphate in the plant with which it is associated and/or provide a source of phosphate to the plant.

Alternatively, or in addition, the endophyte may be capable of nitrogen fixation. Thus, if an endophyte is capable of nitrogen fixation, the plant with which the endophyte is associated may be capable of growing in low nitrogen conditions and/or the endophyte may provide a source of nitrogen to the plant.

In a particularly preferred embodiment, the endophyte provides the ability of the organism to grow in low nitrogen.

As used herein the term “plant of the Poaceae family” is a grass species, particularly a pasture grass such as ryegrass (Lolium) or fescue (Festuca), more particularly perennial ryegrass (Lolium perenne L.) or tall fescue (Festuca arundinaceum, otherwise known as Lolium arundinaceum).

In another aspect, the present invention provides a plant or part thereof infected with an endophyte as hereinbefore described. In preferred embodiments, the plant or part thereof infected with the endophyte may produce a bioprotectant compound, particularly spermidine or derivative, isomer and/or salt thereof. Also in preferred embodiments, the plant or part thereof includes an endophyte-free host plant or part thereof stably infected with said endophyte.

The plant inoculated with the endophyte may be a grass or non-grass plant suitable for agriculture, specifically a forage, turf, or bioenergy grass, or a grain crop or industrial crop.

Preferably, the plant is a grass species plant, specifically a forage, turf, bioenergy, grain crop or industrial crop grass.

The forage, turf or bioenergy grass may be those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens] x Brachiaria brizantha, [Brachiaria ruziziensis x Brachiaria brizantha] x Brachiaria decumbens.

The forage, turf or bioenergy grass may also be those belonging to the genera Lolium and Festuca, including L. perenne (perennial ryegrass) and L. arundinaceum (tall fescue) and L. multiflorum (Italian ryegrass).

The grain crop or industrial crop may be a non-grass species, for example, any of soybeans, cotton and grain legumes, such as lentils, field peas, fava beans, lupins and chickpeas, as well as oilseed crops, such as canola.

Thus, the grain crop or industrial crop species may be slected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, and cotton.

The grain crop or industrial crop grass may be those belonging to the genus Triticum, including T. aestivum (wheat), those belonging to the genus Hordeum, including H. vulgare (barley), those belonging to the genus Avena, including A. sativa (oats), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), and those belonging to the genera Miscanthus, Paspalum, Pennisetum, Poa, Eragrostis and Agrostis.

A plant or part thereof may be infected by a method selected from the group consisting of inoculation, breeding, crossing, hybridisation, transduction, transfection, transformation and/or gene targeting and combinations thereof.

Without wishing to be bound by theory, it is believed that the endophyte of the present invention may be transferred through seed from one plant generation to the next. The endophyte may then spread or locate to other tissues as the plant grows, i.e. to roots.

Alternatively, or in addition, the endophyte may be recruited to the plant root, e.g. from soil, and spread or locate to other tissues.

Thus, in a further aspect, the present invention provides a plant, plant seed or other plant part derived from a plant or part thereof as hereinbefore described. In preferred embodiments, the plant, plant seed or other plant part may produce a bioprotectant compound, particularly spermidine or derivative, isomer and/or salt thereof.

In another aspect, the present invention provides the use of an endophyte as hereinbefore described to produce a plant or part thereof stably infected with said endophyte. The present invention also provides the use of an endophyte as hereinbefore described to produce a plant or part thereof as hereinbefore described.

In another aspect, the present invention provides a bioprotectant compound, produced by an endophyte as hereinbefore described, preferably spermidine or a derivative, isomer and/or a salt thereof.

The bioprotectant compound, preferably spermidine, may be produced by the endophyte when associated with a plant, e.g. a plant of the Poaceae family as described above.

Thus, in another aspect, the present invention provides a method for producing a bioprotectant compound, preferably spermidine, or a derivative, isomer and/or a salt thereof, said method including infecting a plant with an endophyte as hereinbefore described and cultivating the plant under conditions suitable to produce a bioprotectant compound, preferably spermidine.

The endophyte-infected plant or part thereof may be cultivated by known techniques. The person skilled in the art may readily determine appropriate conditions depending on the plant or part thereof to be cultivated.

The bioprotectant compound, preferably spermidine, or a derivative, isomer and/or salt thereof, may also be produced by the endophyte when it is not associated with a plant. Thus, in yet another aspect, the present invention provides a method for producing a bioprotectant compound, preferably spermidine, or a derivative, isomer and/or a salt thereof, said method including culturing an endophyte as hereinbefore described, under conditions suitable to produce the bioprotectant compound.

The conditions suitable to produce the bioprotectant compound may include a culture medium including a source of carbohydrates. The source of carbohydrates may be a starch/sugar-based agar or broth such as potato dextrose agar, potato dextrose broth or half potato dextrose agar or a cereal-based agar or broth such as oatmeal agar or oatmeal broth. Other sources of carbohydrates may include endophyte agar, Murashige and Skoog with 20% sucrose, half V8 juice/half PDA, water agar and yeast malt extract agar. The endophyte may be cultured under aerobic or anaerobic conditions and may be cultured in a bioreactor.

In a preferred embodiment of this aspect of the invention, the method may include the further step of isolating the bioprotectant compound, preferably spermidine or a derivative, isomer and/or a salt thereof, from the plant or culture medium.

The endophyte of the present invention may display the ability to solubilise phosphate.

Thus, in yet another aspect, the present invention provides a method of increasing phosphate use efficiency and/or increasing phosphate solubilisation by a plant, said method including infecting a plant with an endophyte as hereinbefore described, and cultivating the plant.

In yet another aspect, the present invention provides a method of reducing phosphate levels in soil, said method including infecting a plant with an endophyte as hereinbefore described, and cultivating the plant in the soil.

The endophyte of the present invention may be capable of nitrogen fixation. Thus, in yet another aspect, the present invention provides a method of growing the plant in low nitrogen containing medium, said method including infecting a plant with anendophyte as hereinbefore described, and cultivating the plant. Preferably, the low nitrogen medium is low nitrogen containing soil.

In yet a further aspect, the present invention provides a method of increasing nitrogen use efficiency or increasing nitrogen availability to a plant, said method including infecting a plant with an endophyte as hereinbefore described, and cultivating the plant.

In yet another aspect, the present invention provides a method of reducing nitrogen levels in soil, said method including infecting a plant with an endophyte as hereinbefore described, and cultivating the plant in the soil.

In a further aspect, the present invention provides a method of providing bioprotection to a plant against bacterial and/or fungal pathogens and/or providing biofertilizer to a plant, said method including infecting the plant with and endophyte as hereinbefore described. Preferably, the method includes providing bioprotection to the plant and includes production of a bioprotectant compound in the plant into which the endophyte is inoculated.

The endophyte-infected plant or part thereof may be cultivated by known techniques. The person skilled in the art may readily determine appropriate conditions depending on the plant or part thereof to be cultivated.

The production of a bioprotectant compound has particular utility in agricultural plant species, in particular, forage, turf, or bioenergy grass species, or grain crop species or industrial crop species. These plants may be cultivated across large areas of e.g. soil where the properties and biological processes of the endophyte as hereinbefore described and/or bioprotectant compound produced by the endophyte may be exploited at scale.

The part thereof of the plant may be, for example, a seed.

In preferred embodiments, the plant is cultivated in the presence of soil phosphate and/or nitrogen, alternatively or in addition to applied phosphate and/or nitrogen. The applied phosphate and/or applied nitrogen may be by way of, for example, fertiliser. Thus, preferably, the plant is cultivated in soil.

In preferred embodiments, the endophyte may be a Stenotrophomonas rhizophila strain JB as described herein and as deposited with The National Measurement Institute on 17^{th} May 2019 with accession number V19/009906.

Preferably, the plant is a forage, turf, bioenergy grass species or , grain crop or industrial crop species, as hereinbefore described.

The part thereof of the plant may be, for example, a seed.

In preferred embodiments, the plant is cultivated in the presence of soil phosphate and/or applied phosphate. The applied phosphate may be by way of, for example, fertiliser. Thus, preferably, the plant is cultivated in soil.

Alternatively, or in addition, the plant is cultivated in the presence of soil nitrogen and/or applied nitrogen. The applied nitrogen may be by way of, for example, fertiliser. Thus, preferably, the plant is cultivated in soil.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

FIG. 1—16S Amplicon sequence of novel bacterial strain JB (SEQ ID NO: 1).

FIG. 2—Phylogeny of Stenotrophomonas spp. and the novel bacterial strain JB. This maximum-likelihood tree was inferred based on 196 genes conserved among 10 genomes. Values shown next to branches were the local support values calculated using 1000 resamples with the Shimodaira-Hasegawa test.

FIG. 3—Whole genome sequence comparison of the Stenotrophomonas rhizophila novel bacterial strain JB (bottom) and the type Stenotrophomonas rhizophila strain DSM14405 (top). The links between genome sequences indicated percentage similarity (from 70% to 100%). Genetic variations, including non-identical regions, insertions/deletions/inversions and rearrangements, suggest that the novel bacterial strain JB and the bacterial strain DSM14405 are genetically different. The stars represent genomic regions unique to the novel bacterial strain JB or the bacterial strain DSM14405. The triangle represents genomic regions with 70% sequence homology between the novel bacterial strain JB or the bacterial strain DSM14405. The square represents genomic regions that have undergone rearrangement.

FIG. 4—Bioprotection bioassay indicating the growth of 11 strains (including the S. rhizophila novel bacterial strain JB, star) against 6 μlant pathogenic fungi, Fusarium verticillioides (10 days post inoculation, dpi), Bipolaris gossypina (7 dpi), Sclerotinia rolfsii (5 dpi), Drechslera brizae (8 dpi), Phoma sorghina (9 dpi) and Microdochium nivale (6 dpi). Bars represent the mean diameter of fungal colonies from three replicate plates of each treatment. Different superscript letters indicate significant differences (P<0.05) between treatments.

FIG. 5—Secondary metabolite biosynthesis gene clusters in the Stenotrophomonas rhizophila novel bacterial strain JB identified using antiSMASH (Weber et al. 2015). The gene clusters have sequence homology and structure to (A) a bacteriocin-like gene cluster; (B) a lantipepetide-like gene cluster; (C) an unknown NRPS gene cluster; (D) an arylpolyene-like gene cluster. The core biosynthetic genes of each cluster are designated by a black line.

FIG. 6—Biofertiliser activity (in vitro) of the Stenotrophomonas rhizophila novel bacterial strain JB and other bacterial strains on semi-solid NfB medium, which determines the ability of bacteria to grow under low N. A) Absorbance readings across a wavelength range of 300-800 nm (615 nm-optimal wavelength for bioassay) for 8 bacterial strains and a no growth control (NGC—NfB media only). B) Growth of 8 bacterial strains and a NGC in semi-solid NfB media in a 96 well plate, indicating strains capable of growing under low N (dark—strains 2, 3, 4, 5, 6, JB) and those strains that cannot (light—1, 7, -ve control,

NGC).

FIG. 7—Gene clusters of Stenotrophomonas rhizophila (strains JB and DSM14405) responsible for the regulation of the important plant polyamine spermidine. The spermidine synthase is designated by a start, while the triangle designates regions that differ between the two strains.

FIG. 8—Image of 5 day old seedlings inoculated with the Stenotrophomonas rhizophila novel bacterial strain JB and an untreated control (blank).

FIG. 9—Average shoot and root length of barley seedlings inoculated with the Stenotrophomonas rhizophila novel bacterial strain JB and an untreated control (blank), and grown for 5 days. There was no significant difference (p-value<0.05) between the two treatments.

FIG. 10—Average root length of barley seedlings inoculated with bacterial strains of Stenotrophomonas rhizophila (strain JB) and non-Stenotrophomonas strains (Strain 1, 2, 3, 4) and grown for 4 days on media containing insoluble phosphate. The star indicates significant difference in the mean at p 0.05 between the control and the bacterial strains.

FIG. 11—Average shoot length of barley seedlings inoculated with bacterial strains of Stenotrophomonas rhizophila (strain JB) and non-Stenotrophomonas strains (Strain 1, 2, 3, 4) and grown for 4 days on media containing insoluble phosphate. The star indicates significant difference in the mean at p 0.05 between the control and the bacterial strains.

### DETAILED DESCRIPTION OF THE EMBODIMENTS

Isolation and Characterisation of Plant Associated Stenotrophomonas rhizophila Novel Bacterial Strains Providing Bioprotection and Biofertilizer Phenotypes to Plants.

The novel plant associated Stenotrophomonas rhizophila bacterial strain JB has been isolated from perennial ryegrass (Lolium perenne) plants. It displays the ability to inhibit the growth of plant fungal pathogens and an ability to grow in low nitrogen in plate assays. The genome of the Stenotrophomonas rhizophila bacterial strain JB has been sequenced and is shown to be novel, related to bioprotectant Stenotrophomonas rhizophila strains and not pathogenic Stenotrophomonas maltophilia strains. Analysis of the genome sequence has shown that the Stenotrophomonas rhizophila novel bacterial strain JB has gene clusters for the biosynthesis of the antibacterial and antifungal bioprotectant compounds and genes involved in plant growth/endophytic niche via the production of spermidine. This novel bacterial strain has been used to inoculate barley (Hordeum vulgare) seeds under glasshouse conditions and has been demonstrated not to cause disease in these barley plants. These barley plants are also able to produce seed. Novel bacterial strain JB also enhances root and shoot growth in insoluble phosphate. Overall, novel plant associated Stenotrophomonas rhizophila bacterial strain JB offer both bioprotectant and biofertilizer activity.

### EXAMPLE 1

### Isolation of Bacterial Strains

Seed Associated Bacterial Strains

Seeds from perennial ryegrass (Lolium perenne) were surface-sterilised by soaking in 80% ethanol for 3 mins, then washing 5 times in sterile distilled water. The seeds were then plated onto sterile filter paper soaked in sterile water in sterile petri dishes. These plates were stored at room temperature in the dark to allow seedlings to germinate for 1-2 weeks. Once the seedlings were of sufficient size, the plants were harvested. In harvesting, the remaining seed coat was discarded, and the aerial tissue and root tissue were harvested. The plant tissues were submerged in sufficient Phosphate Buffered Saline (PBS) to completely cover the tissue, and ground using a Qiagen TissueLyser II, for 1 minute at 30

Hertz. A 10 μl aliquot of the macerate was added to 90 μl of PBS. Subsequent 1 in 10 dilutions of the 10⁻¹ suspension were used to create additional 10⁻² to 10⁻⁴ suspensions. Once the suspensions were well mixed 50 μl aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony.

Mature Plant Associated Bacterial Strains

Leaf and root tissue were harvested from mature plants grown in the field or grown in pots in a greenhouse. Root tissue was washed in PBS buffer to remove soil particles and sonicated (10 mins) to remove the rhizosphere. The harvested tissues were placed into sufficient PBS to completely cover the tissue and processed as per the previous section to isolate pure bacterial cultures.

Around 300 bacterial strains were obtained from seeds of perennial ryegrass, and 300 strains from mature perennial ryegrass plants. The novel bacterial strain JB was collected from seed of perennial ryegrass.

### EXAMPLE 2

### Identification of Stenotrophomonas rhizophila Novel Bacterial Strain

Amplicon (16S rRNA gene) Sequencing

A phylogenetic analysis of the novel bacterial strain JB was undertaken by sequence homology comparison of the 16S rRNA gene. The novel bacterial strain JB was grown overnight in Reasoners 2 Broth (R2B) media. DNA was extracted from pellets derived from the overnight culture using a DNeasy Blood and Tissue kit (Qiagen) according to manufacturer's instructions. The 16S rRNA gene amplification used the following PCR reagents: 14.8 μL H₂O, 2.5 μL 10× reaction buffer, 0.5 μL 10 mM dNTPs, 2.5 μL each of the 5 μM 27F primer (5′-AGAGTTTGATCMTGGCTCAG-3′) (SEQ ID NO 2) and 5 μM reverse primers 1492R (5′-GGTTACCTTGTTACGACTT-3′) (SEQ ID NO: 3), 0.2 μL of Immolase enzyme, and template to a final volume of 25 μL. The PCR reaction was then run in an Agilent Surecycler 8800 (Applied Biosystems) with the following program; a denaturation step at 94° C. for 15 min; 35 cycles of 94° C. for 30 sec, 55° C. for 10 sec, 72° C. 1 min; and a final extension step at 72° C. for 10 min.

Shrimp alkaline phosphatase (SAP) exonuclease was used to purify the 16S rRNA gene PCR amplicon. The SAP amplicon purification used the following reagents: 7.375 μL H₂O, 2.5 μL 10× SAP, and 0.125 μL Exonuclease I. The purification reaction was incubated at 37° C. for 1 hr, followed by 15 min at 80° C. to deactivate the exonuclease.

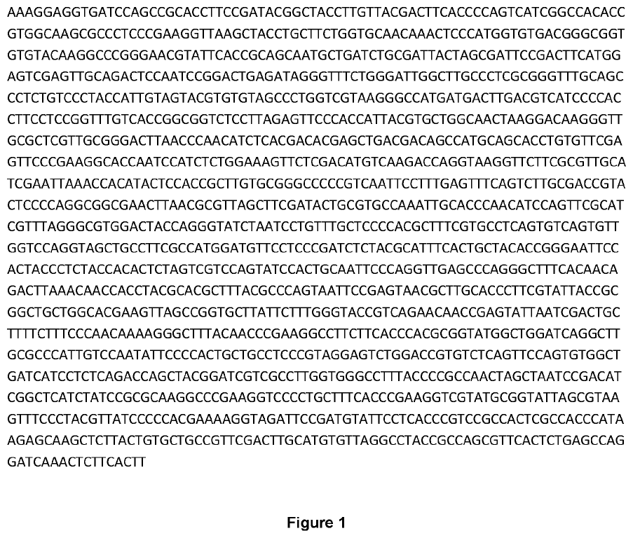

The purified 16S rRNA gene amplicon was sequenced using the BigDye® Terminator v3.1 Cycle Sequencing Kit (Thermofisher) with the following reagents; 10.5 μL H₂O, 3.5 μL 5× Seq buffer, 0.5 μL BigDye®, 2.5 μL of either the 3.2 μM Forward (27F) and 3.2 μM Reverse primers (1492R), and 4.5 μL of PCR amplicon as template, to a final reaction volume of 20 μL. The sequencing PCR reaction was then run in an Agilent Surecycler 8800 (Applied Biosystems) with the following program; denaturation step at 94° C. for 15 min; followed by 35 cycles of 94° C. for 30 sec, 55° C. for 10 sec, 72° C. 1 min; and one final extension step at 72° C. for 10 min. The 16S rRNA gene amplicon from novel bacterial strain JB was sequenced on an AB13730XL (Applied Biosystems). A 1546 bp 16S rRNA gene sequence was generated (FIG. 1). The sequence was aligned by BLASTn on NCBI against the non-redundant nucleotide database and the 16S ribosomal RNA database.

BLASTn Hit Against Database nr; Stenotrophomonas rhizophila Strain e-p10 16S Ribosomal RNA Gene, Partial Sequence t,?

BLASTn Hit Against Database 16S Ribosomal RNA; Stenotrophomonas rhizophila Strain e-p10 16S Ribosomal RNA Gene, Partial Sequence t,?

The preliminary taxonomic identification of the novel bacterial strain JB was Stenotrophomonas rhizophila.

Genomics

The genome of the Strenotrophomonas rhizophila novel bacterial strain JB was sequenced. This novel bacterial strain was retrieved from the glycerol collection stored at −80° C. by streaking on R2A plates. Single colonies from these plates were grown overnight in Nutrient Broth and pelleted. These pellets were used for genomic DNA extraction using the bacteria protocol of Wizard® Genomic DNA Purification Kit (A1120, Promega). A DNA sequencing library was generated for Illumina sequencing using the Illumina Nextera XT DNA library prep protocol. The library was sequenced using an Illumina MiSeq platform. Raw reads from the sequencer were filtered to remove any adapter and index sequences as well as low quality bases using Trimmomatic (Bolger, Lohse & Usadel 2014) with the following options: ILLUMINACLIP: NexteraPE-PE.fa:2:30:10 LEADING:3 TRAILING:3 SLIDINGWINDOW:4:15 MINLEN:36. To enable full genome assembly, long reads were generated for novel bacterial strain JB by sequencing DNA using Oxford Nanopore Technologies (ONT) MinION platform. The DNA from the Wizard® Genomic DNA Purification Kit was first assessed with the genomic assay on Agilent 2200 TapeStation system (Agilent Technologies, Santa Clara, Calif., USA) for integrity (average molecular weight 30 Kb). The sequencing library was prepared using an in-house protocol modified from the official protocols for transposases-based library preparation kits (SQK-RAD004/SQK-RBK004, ONT, Oxford, UK). The library was sequenced on a MinION Mk1B platform (MIN-101B) with R9.4 flow cells (FLO-MIN106) and under the control of MinKNOW software. After the sequencing run finished, the fast5 files that contain raw read signals were transferred to a separate, high performance computing Linux server for local basecalling using ONT's Albacore software (Version 2.3.1) with default parameters. The sequencing summary file produced by Albacore was processed by the R script minion qc (https://github.com/roblanf/minion_qc) and NanoPlot (De Coster et al. 2018) to assess the quality of the sequencing run, while Porechop (Version 0.2.3, https://github.com/rrwick/Porechop) was used to remove adapter sequences from the reads. Reads which were shorter than 300 bp were removed and the worst 5% of reads (based on quality) were discarded by using Filtlong (Version 0.2.0, https://github.com/rrwick/Filtlong).

The whole genome sequence of novel bacterial strain JB was assembled using Unicycler (Wick et al. 2017). Unicycler performed hybrid assembly when both Illumina reads and

MinION reads were available. MinION reads were mainly used to resolve repeat regions in the genome sequence, whereas Illumina reads were used by Pilon (Walker et al. 2014) to correct small base-level errors. Multiple rounds of Racon (Vaser et al. 2017) polishing were then carried out to generate consensus sequences. Assembly graphs were visualised by using Bandage (Wick et al. 2015).

A complete circular chromosome sequence was produced for the novel bacterial strain JB. The genome size for the novel bacterial strain JB was 4,667,358 bp (Table 1). The percent GC content was 67.27%. The novel bacterial strain JB was annotated by Prokka (Seemann 2014) with a custom, genus-specific protein database to predict genes and corresponding functions, which were then screened manually to identify specific traits. The number of genes for the novel bacterial strain JB was 4,141 (Table 2).

Table 1—Summary of Properties of the Final Genome Sequence Assembly

TABLE 1
Summary of properties of the final genome sequence assembly

| | Genome size | GC content | Coverage | Coverage |
|---|---|---|---|---|
| Strain ID | (bp) | (%) | Illumina reads | ONT MinION |
| JB | 4,667,358 | 67.27 | 698× | 72× |

Table 2—Summary of Genome Coding Regions

TABLE 2
Summary of genome coding regions

| Strain | Genome | No. of | No. of | No. of | No. of | No. of |
|---|---|---|---|---|---|---|
| ID | size (bp) | tRNA | tmRNA | rRNA | CDS | gene |
| JB | 4,667,358 | 74 | 1 | 10 | 4,056 | 4,141 |

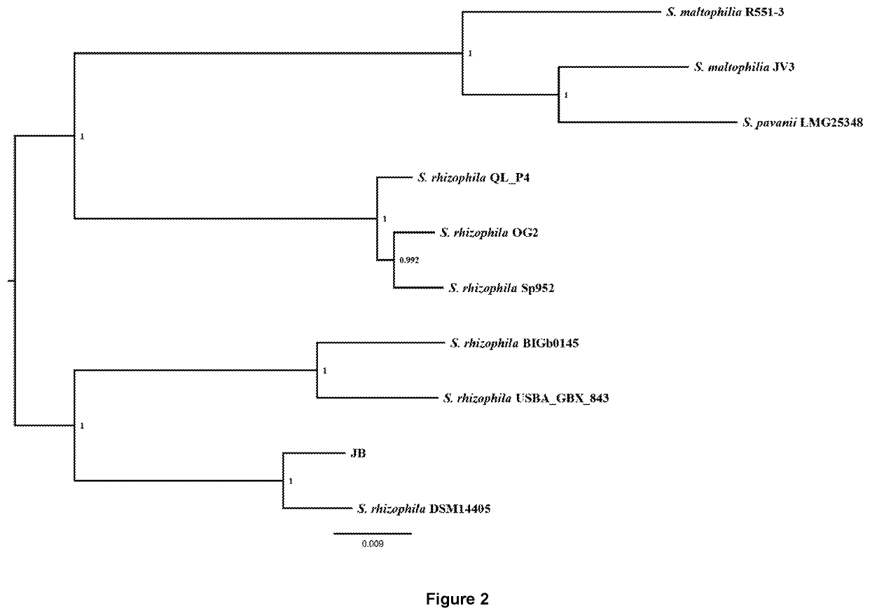

Nine Stenotrophomonas spp. (S. rhizophila, S. maltophilia, S. pavanii) genome sequences that are publicly available on NCBI were acquired and used for pan-genome/comparative genome sequence analysis alongside the novel bacterial strain JB. A total of 196 genes that are shared by all 10 strains were identified by running Roary (Page et al. 2015). PRANK (Loytynoja 2014) was then used to perform a codon aware alignment. A maximum-likelihood phylogenetic tree (FIG. 2) was inferred using FastTree (Price, Dehal & Arkin 2010) with Jukes-Cantor Joins distances and Generalized Time-Reversible and CAT approximation model. Local support values for branches were calculated using 1000 resamples with the Shimodaira-Hasegawa test. The novel bacterial strain JB clustered tightly with the bioprotectant S. rhizophila strain DSM14405 (type strain of this species), suggesting a close phylogenetic relationship between these two bacterial strains. Moreover, this cluster was separated from other Stenotrophomonas spp. with strong local support value (100%), including the human pathogen S. maltophilia. This separation supports that bacterial strain JB is novel and from the species S. rhizophila.

The average nucleotide identity (ANI) was calculated for novel bacterial strain JB against the other nine Stenotrophomonas spp. strains (Table 3). The genome sequences of the ten strains were aligned and compared using minimap2 (Li 2018). Based on a species boundary of 95-96% (Chun et al. 2018; Richter & Rosselló-Móra 2009) the bacterial strain JB is from S. rhizophila, but is novel and a different strain to the type strain of this species (DSM14405) (Wolf et al. 2002).

Table 3—Average Nucleotide Identity (ANI) of Ten Strains of Stenotrophomonas spp. Including Novel Bacterial Strain JB and the Type S. rhizophila Strain DSM14405

TABLE 3
Average nucleotide identity (ANI) of ten strains of Stenotrophomonas spp.
including novel bacterial strain JB and the type S. rhizophila strain DSM14405

| | | S. | | S. | | | S. | | | | |
|---|---|---|---|---|---|---|---|---|---|---|---|
| | | rhizo- | S. | rhizophila | S. | S. | rhizo- | S. | | S. | S. |
| | | phila | rhizophila | USBA_ | rhizophila | rhizophila | phila | rhizophila | S. pavanii | maltophilla | maltophilla |
| | | JB | DSM14405 | GBX_843 | BIGb0145 | Sp952 | OG2 | QL_P4 | LMG25348 | JV3 | R551-3 |
| S. rhizophila | JB | | 96.44% | 86.19% | 86.33% | 86.12% | 86.13% | 86.12% | 82.73% | 82.76% | 82.57% |
| S. rhizophila | DSM14405 | | | 86.41% | 86.33% | 86.25% | 86.21% | 86.19% | 82.77% | 82.83% | 82.63% |
| S. rhizophila | USBA_ | | | | 93.41% | 85.21% | 85.23% | 85.21% | 82.47% | 82.51% | 82.34% |
| | GBX_843 | | | | | | | | | | |
| S. rhizophila | BIGb0145 | | | | | 85.08% | 85.13% | 85.07% | 82.30% | 82.46% | 82.18% |
| S. rhizophila | Sp952 | | | | | | 97.78% | 97.43% | 82.86% | 82.90% | 82.62% |
| S. rhizophila | OG2 | | | | | | | 97.74% | 82.83% | 82.88% | 82.60% |
| S. rhizophila | QL_P4 | | | | | | | | 82.78% | 82.92% | 82.61% |
| S. pavanii | LMG25348 | | | | | | | | | 92.35% | 90.28% |
| S. maltophilia | JV3 | | | | | | | | | | 91.27% |
| S. maltophilia | R551-3 | | | | | | | | | | |

Genome Sequence Alignment

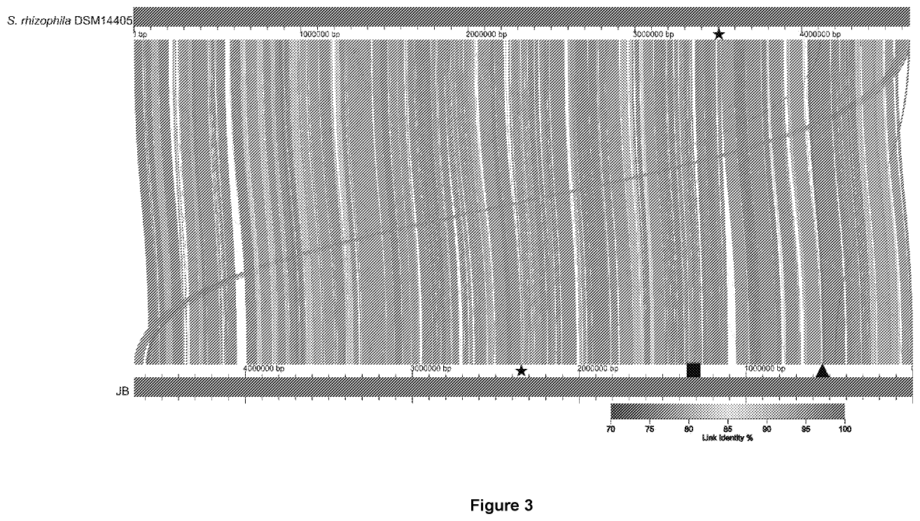

The genome sequences of Stenotrophomonas rhizophila strain JB and the type strain DSM14405 were aligned using LASTZ (Version 1.04.00, http://www.bx.psu.edu/-rsharris/lastz/) and visualised using AliTV (Ankenbrand et al. 2017) to determine the genomic similarity between the two strains. The genomes of the two strains were similar, but there were large genomic regions unique to the novel bacterial strain JB or the bacterial strain DSM14405 (FIG. 3—stars). Similarly, there are a large number of genomic regions that have undergone rearrangements (FIG. 3—square) or have low sequence homology (e.g. 70% homology, FIG. 3—triangle).

### EXAMPLE 3

### Bioprotection Activity (In Vitro) of the Stenotrophomonas rhizophila Novel Bacterial Strain JB

In vitro bioassays were established to test the bioactivity of 11 μlant associated bacterial strains including Stenotrophomonas rhizophila novel bacterial strain JB, against six plant pathogenic fungi (Table 4). A plate with only the pathogen was used as a negative control (blank). The fungal pathogens were all isolated from monocot species, and were obtained from the National Collection of Fungi (Herbarium VPRI) and the AVR collection. Each bacterial strain was cultured in Nutrient Broth (BD Biosciences) overnight at 28° C. in a shaking incubator (200 rpm). Each bacterial strain was drop-inoculated (20 μL) onto four equidistant points on a Nutrient Agar (BD Biosciences) plate, which was then incubated overnight at 28° C. A 6mm×6mm agar plug of actively growing mycelia from the pathogen was placed at the centre of the plate. The bioassay was incubated for at least 5 days at 28° C. in the dark, and then the diameter of the fungal colony on the plate was recorded. For each treatment three plates were prepared as biological triplicates. OriginPro 2018 (Version b9.5.1.195) was used to carry out One-way ANOVA and Tukey Test to detect the presence of any significant difference (p 0.05) between treatments.

Table 4—Pathogens Used in the Bioprotection Bioassay.

TABLE 4
Pathogens used in the bioprotection bioassay.

| Accession | | Taxonomic | | Collection |
|---|---|---|---|---|
| No. | VPRI | Taxonomic Details | Host | |
| 12962 | Drechslera brizae (Y.Nisik.) Subram. | Briza maxima L. | Vic. | Oct. 24, 1985 |
| | & B.L.Jain | | | |
| 32148 | Sclerotium rolfsii Sacc. | Poa annua L. | Vic. | Jan. 1, 2005 |
| 10694 | Phoma sorghina (Sacc.) Boerema, | Cynodon | Vic. | Apr. 19, 1979 |
| | Dorenbosch, van Kesteren | dactylon Pers. | | |
| 42586a | Fusarium verticillioides (Sacc.) | Zea mays L. | Vic. | Feb. 27, 2015 |
| | Nirenberg | | | |
| 42563 | Bipolaris gossypina | Brachiaria | Qld | |
| N/A | Microdochium nivale | Lolium perenne | Vic | |
| | | L. | | |

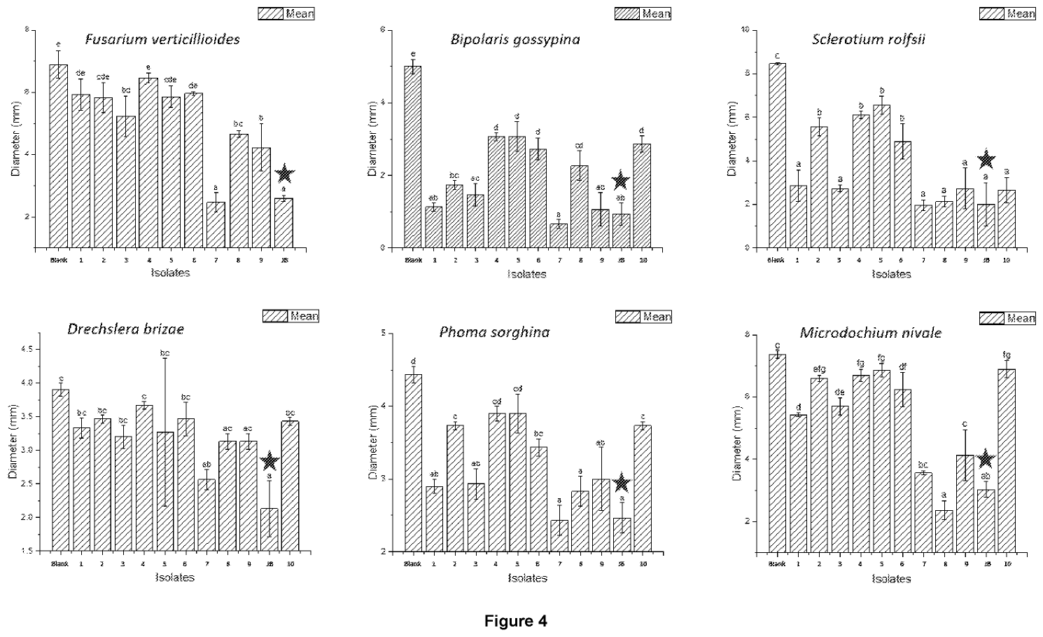

The Stenotrophomonas rhizophila novel bacterial strain JB inhibited the growth of all six fungal pathogens compared to the control and many of the other test bacterial strains, indicating it had broad spectrum biocidal activity (FIG. 4). The S. rhizophila novel bacterial strain JB was the most active bacterial strain against Drechslera brizae, while it was the second most active strain against Fusarium verticillioides, Bipolaris gossypina, Sclerotium rolfsii, Phoma sorghina and Microdochium nivale.

### EXAMPLE 4

### Genome Sequence Features Supporting the Bioprotection Niche of the Stenotrophomonas rhizophila Novel Bacterial Strain JB

Secondary Metabolite Biosynthesis Gene Clusters

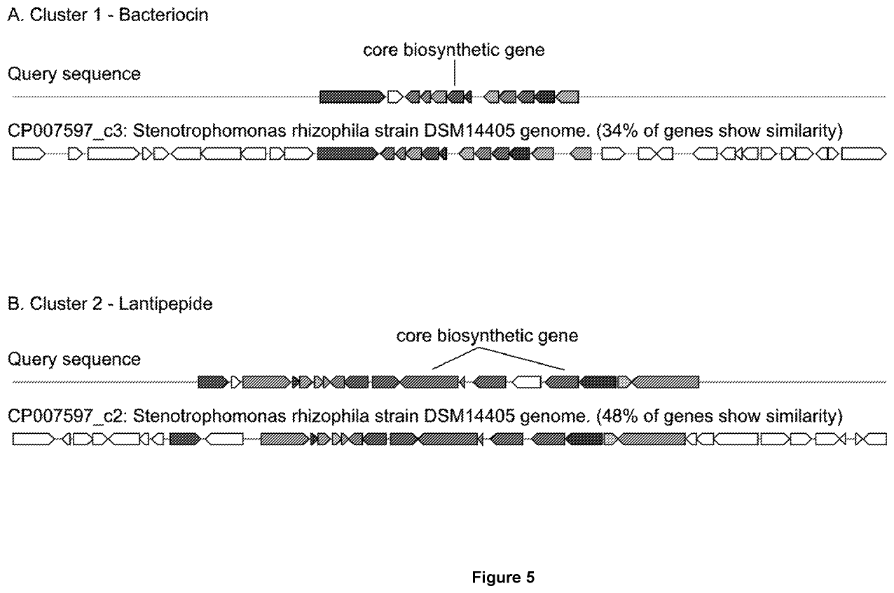

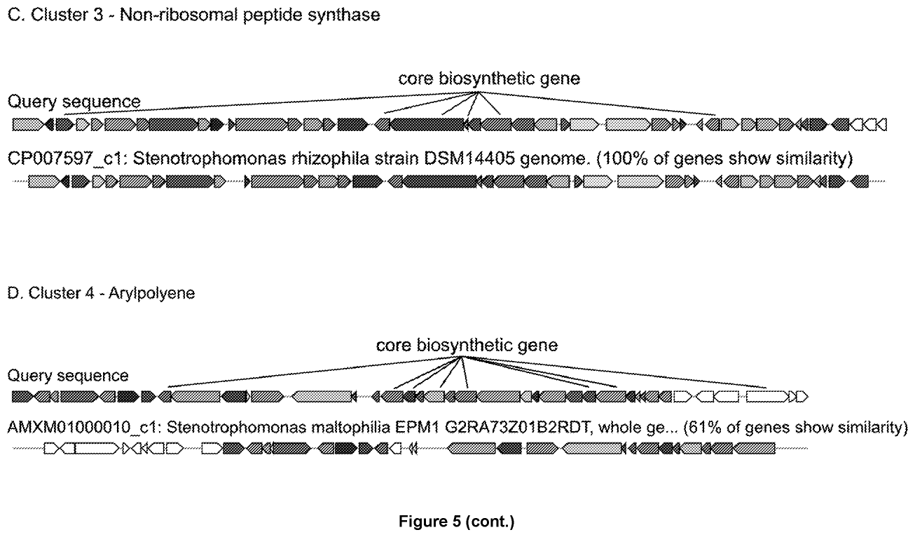

The genome sequence of the Stenotrophomonas rhizophila novel bacterial strain JB was assessed for the presence of features associated with bioprotection. The annotated genome was analysed by antiSMASH (Weber et al. 2015) to identify secondary metabolite biosynthesis gene clusters that are commonly associated with the production of biocidal compounds that aid in their defence. An annotated genome was passed through antiSMASH with the following options: —clusterblast—asf—knownclusterblast—subclusterblast—smcogs—full-hmmer. A total of four secondary metabolite gene clusters were identified in the genome sequence of the Stenotrophomonas rhizophila novel bacterial strain JB. (FIG. 5A-D). These included a bacteriocin-like gene cluster (cluster 1), a lantipeptide-like (bacteriocin) gene cluster (cluster 2), an unknown non-ribosomal peptide synthase (NRPS) gene cluster (cluster 3), and an arylpolyene-like gene cluster (cluster 4). Cluster 1 had one core biosynthetic gene and showed 34% similarity to a cluster in the genome sequence of the type strain of Stenotrophomonas rhizophila (DSM14405) (FIG. 5A). Cluster 2 had two core biosynthetic genes and showed 48% similarity to a cluster in the genome sequence of the type strain of Stenotrophomonas rhizophila (DSM14405) (FIG. 5B). Cluster 3 had six core biosynthetic genes and showed 100% similarity to a cluster in the genome sequence of the type strain of Stenotrophomonas rhizophila (DSM14405) (FIG. 5C). Cluster 4 had eight core biosynthetic genes and showed 61% similarity to a cluster in the genome sequence of Stenotrophomonas maltophilia (EPM1 G2RA73Z01B2RDT) (FIG. 5D). The proposed function of clusters 1 and 2 is thought to involve the biosynthesis of bacteriocins, which have antimicrobial activity against similar or closely-related bacterial strains. The proposed function of cluster 3 is unclear. The proposed function of cluster 4 is thought to involve the biosynthesis of an arylpolyene, some of which have antimicrobial activity against fungi.

### EXAMPLE 5

### Biofertiliser Activity (In Vitro) of the Stenotrophomonas rhizophila Novel Bacterial Strain JB

Nitrogen (N) is an important nutrient for plant growth and a key component of fertilisers. Plant associated bacteria able to grow under low nitrogen conditions may be useful in plant growth as the bacteria can pass this N onto the plant. This was assessed by using the nitrogen-free NFb medium (Dobereiner 1980). One litre of NFb medium contains 5 g DL-malic acid, 0.5 g dipotassium hydrogen orthophosphate, 0.2 g magnesium sulfate heptahydrate, 0.1 g sodium chloride, 0.02 g calcium chloride dehydrate, 2 mL micronutrients solution [0.4 g/L copper sulfate pentahydrate, 0.12 g/L zinc sulfate heptahydrate, 1.4 g/L boric acid, 1 g/L sodium molybdate dehydrate, 1.5 g/L manganese(II) sulfate monohydrate], 1 mL vitamin solution (0.1 g/L biotin, 0.2 g/L pyridoxol hydrochloride), 4 mL iron(III) EDTA and 2 mL bromothymol blue (0.5%, dissolved in 0.2N potassium hydroxide). For solid NFb medium, 15 g/L bacteriological agar was added, otherwise 0.5 g/L was added for semi-solid medium. The pH of medium was adjusted to 6.8. To detect the nitrogen fixation ability, bacterial strains were inoculated onto solid medium plates. For each inoculation, triplicates were prepared. All NFb medium plates were incubated at 30° C. After 96 hours, the colour change of NFb medium plates was recorded, with development of blue colour an indication of growth under limiting N.

In the high throughput automated method to detect nitrogen fixation ability semi-solid media NfB was used. Bacterial strains were inoculated into 20 mL R2B medium (0.5 g/L yeast extract, 0.5 g/L proteose peptone, 0.5 g/L casein hydrolysate, 0.5 g/L glucose, 0.5 g/L starch, 0.3 g/L dipotassium hydrogen orthophosphate, 0.024 g/L magnesium sulphate and 0.3g/L sodium pyruvate) and incubated at 28° C. and 200rpm overnight. The cell pellet was collected by centrifuging at 4000×g for 3 minutes, and then was twice with 1XPBS to remove the nitrogen residue from R2B. Then cell pellet was resuspended in 10 mL semi-solid NFb medium. 1 μL of cell suspension was added to a well containing 199 μL semi-solid NFb medium on a 96-well cell culture plate. For each strain, cell suspension was added to six consecutive wells of the same column, representing six biological replicates. Wells that are located in row A and H, and column 1 and 12 were excluded during the examination due to the edge effect which may lead to unreliable reading. The plate was incubated at room temperature for 27 hours, after which the plate was examined by the plate reader by conducting a spectrum scan (300 nm-750 nm wavelength, 10 nm increment). An increase in absorbance represented an increase in growth under low N conditions.

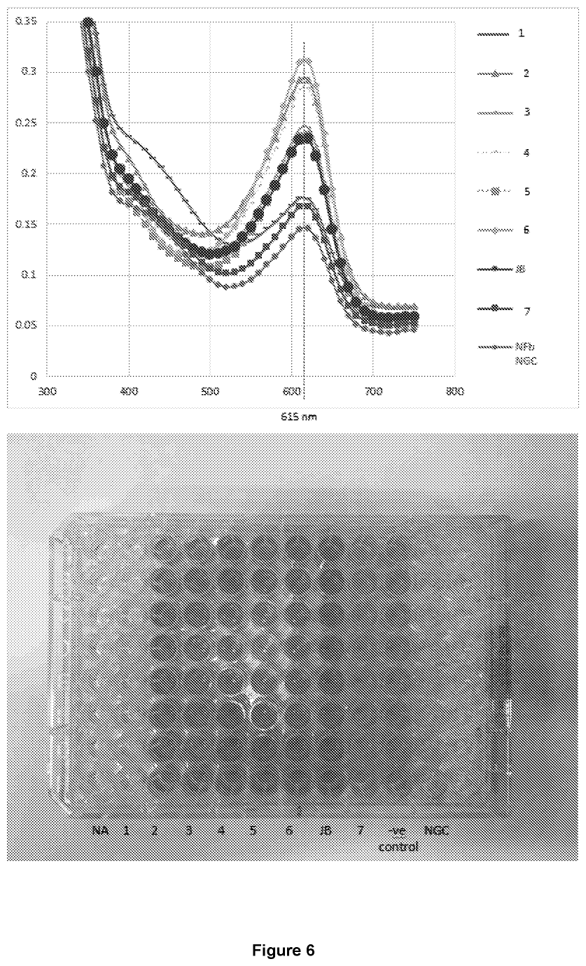

The Stenotrophomonas rhizophila novel bacterial strain JB was able to grow under low N, as evident from the colour change in the NfB media and elevated absorbance levels at a wavelength of 615 nm (FIG. 6A and 6B).

### EXAMPLE 6

### Genome Sequence Features Supporting the Endophytic Niche of the Stenotrophomonas rhizophila Novel Bacterial Strain JB

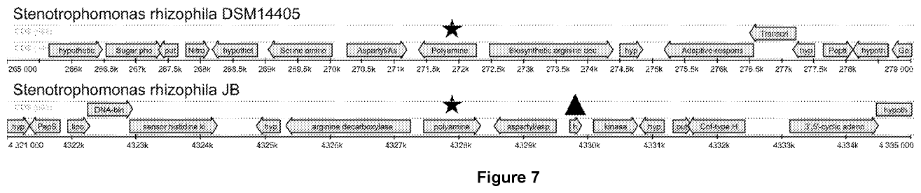

Spermidine is an important polyamine involved in seed and embryo development, regulation of plant growth (particularly roots), and tolerance against drought and salinity (Gill & Tuteja 2010; Hummel et al. 2002; Imai et al. 2004). The biosynthesis of spermidine is regulated by spermidine synthases that catalyse the production of spermidine from putrescine and decarboxylated S-adenosylmethionine (dcSAM). Spermidine synthases have been identified in plant associated bacteria including Stenotrophomonas rhizophila and have been shown to be critical for the plant growth promotion activity of the bacterium (Alavi et al. 2014; Xie et al. 2014). The genome sequence of the Stenotrophomonas rhizophila novel bacterial strain JB was analysed and a spermidine synthase gene was identified (FIG. 7). The gene showed 100% sequence homology to a complementary gene in the genome of the type Stenotrophomonas rhizophila strain DSM14405 (FIG. 7—stars), however the surrounding genes showed significant variability including the addition of a hypothetical gene (FIG. 7—triangle).

### EXAMPLE 7

### In Planta Inoculations Supporting Endophytic Niche of the Stenotrophomonas rhizophila Novel Bacterial Strain JB

To assess direct interactions between the Stenotrophomonas rhizophila novel bacterial strain JB and plants, an early seedling growth assay was established in barley (Hordeum vulgare). The Stenotrophomonas rhizophila novel bacterial strain JB was cultured in

Lysogeny Broth (LB) overnight at 26° C. The following day seeds of barley (cultivar Hindmarsh) were surface-sterilised by soaking in 80% ethanol for 3 mins, then washing 5 times in sterile distilled water. The seeds were then soaked in the overnight cultures for 4 hours at 26° C. in a shaking incubator. For control seedlings, seeds were soaked in LB without bacteria for 4 hours at 26° C. in a shaking incubator. The seeds were planted into a pot trial, with three replicates (pots) per strain/control, with a randomised design. A total of 20 seeds were planted per pot, to a depth of 1 cm. The potting medium contained a mixture of 25% potting mix, 37.5% vermiculite and 37.5% perlite. The plants were grown for 5 days and then removed from the pots, washed, assessed for health (i.e. no disease symptoms) and photographed. The lengths of the longest root and the longest shoot were measured. Data was statistically analysed using a t test to detect the presence of any significant difference (p 0.05) between treatments using Excel.

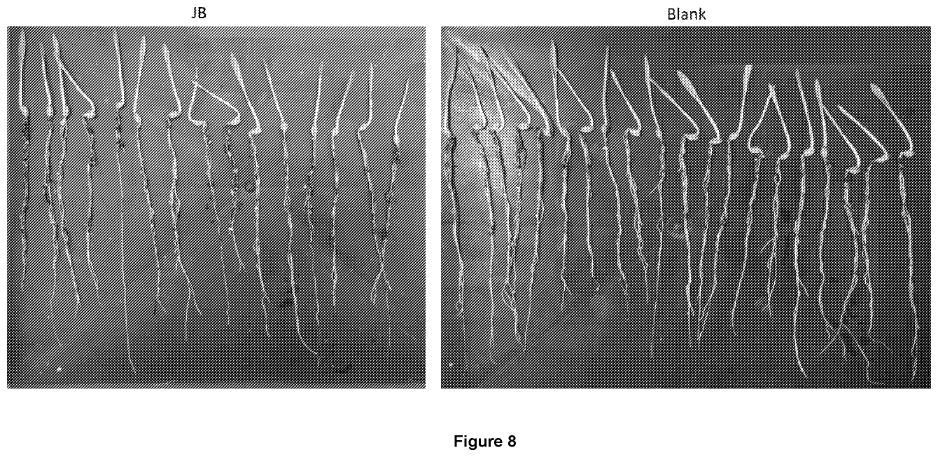

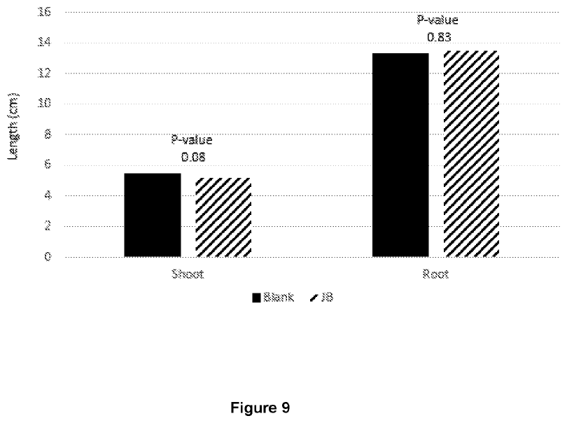

Seedlings inoculated with the Stenotrophomonas rhizophila novel bacterial strain JB were healthy with no disease symptoms recorded on leaves or roots (FIG. 8). The length of the shoots inoculated with the Stenotrophomonas rhizophila novel bacterial strain JB were equivalent to the control (FIG. 9).

### EXAMPLE 8

### In Planta Inoculations Supporting the Biofertilizer (Phosphate Solubilisation) Niche of the Stenotrophomonas rhizophila Novel Bacterial Strain JB

An in planta biofertilizer assay was established in barley to evaluate the ability of Stenotrophomonas rhizophila novel bacterial strain JB to aid growth under conditions with insoluble phosphate. Initially, bacterial strains (5, including JB) were cultured in 30 mL R2B overnight at 26° C. whilst rotating at 200 RPM. The following day the barley seeds were sterilized in 70% ethanol for 5 minutes, followed by rinsing with SDW for five times. These sterile seeds were submerged in the overnight cultures for 4 hours in a dark incubator at room temperature whilst rotating at 200 RPM. The seeds were subsequently transferred to moistened sterile filter paper to be allowed to germinate for three days. These three-day-old seedlings were individually transferred to 60 mm plates with semi-solid Pikovskaya media which contains yeast extract (0.5 g/L), D-glucose (5.0 g/L), calcium phosphate (5.0 g/L), ammonium sulphate (0.5 g/L), potassium chloride (0.2 g/L), magnesium sulphate (0.1 g/L), manganese sulphate (0.1 mg/L), ferrous sulphate (0.1 mg/L) and agar (5.0 g/L). These seedlings were allowed to grow for another 4 days, before the shoots and roots were measured for each seedling. There was a total of 6 treatments (5 bacterial strains including JB; 1 blank media control) containing 10 seedlings per treatment. Statistical analysis (One-way ANOVA and Tukey Test) was conducted using OriginPro 2018 (Version b9.5.1.195) to detect the presence of any significant difference (P<0.05) between treatments.

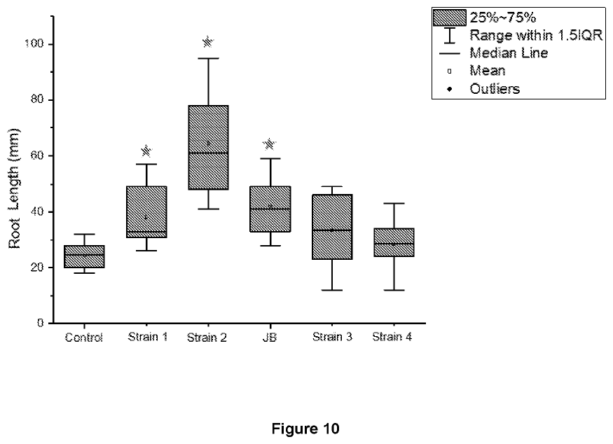

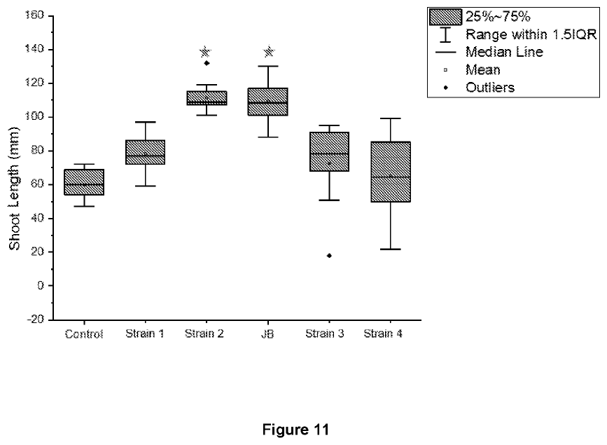

The root growth of seedlings inoculated with novel bacterial strain JB and grown under conditions with insoluble phosphate was significantly greater than the control (P<0.05), with an average increase of 42.6% (FIG. 10). The shoot growth of seedlings inoculated with novel bacterial strain JB was significantly greater than the control (P<0.05), with an average increase of 45.2% (FIG. 11). Overall, results indicate that novel bacterial strain JB can aid in the growth of seedlings grown under conditions with insoluble phosphate.

It is to be understood that various alterations, modifications and/or additions may be made without departing from the spirit of the present invention as outlined herein.

As used herein, except where the context requires otherwise, the term “comprise” and variations of the term, such as “comprising”, “comprises” and “comprised”, are not intended to be in any way limiting or to exclude further additives, components, integers or steps.

Reference to any prior art in the specification is not, and should not be taken as, an acknowledgment or any form of suggestion that this prior art forms part of the common general knowledge in Australia or any other jurisdiction or that this prior art could reasonably be expected to be combined by a person skilled in the art.

### REFERENCES

1. Alavi, P, Starcher, M R, Thallinger, G G, Zachow, C, Müller, H & Berg, G 2014, ‘Stenotrophomonas comparative genomics reveals genes and functions that differentiate beneficial and pathogenic bacteria’, BMC genomics, vol. 15, no. 1, p. 482.

2. Ankenbrand, M J, Hohlfeld, S, Hackl, T & Förster, F 2017, ‘AliTV—interactive visualization of whole genome comparisons’, PeerJ Computer Science, vol. 3.

3. Bolger, A M, Lohse, M & Usadel, B 2014, ‘Trimmomatic: a flexible trimmer for Illumina sequence data’, Bioinformatics, vol. 30, no. 15, pp. 2114-20.

4. Chun, J, Oren, A, Ventosa, A, Christensen, H, Arahal, D R, da Costa, M S, Rooney, A P, Yi, H, Xu, XW, De Meyer, S & Trujillo, ME 2018, ‘Proposed minimal standards for the use of genome data for the taxonomy of prokaryotes’, Int J Syst Evol Microbiol, vol. 68, no. 1, pp. 461-6.

5. De Coster, W, D'Hert, S, Schultz, D T, Cruts, M & Van Broeckhoven, C 2018, ‘NanoPack: visualizing and processing long read sequencing data’, Bioinformatics.

6. Dobereiner, J 1980, ‘Forage grasses and grain crops’, in Methods for evaluating biological nitrogen fixation/edited by FJ Bergersen, John Wiley & Sons Ltd, pp. 535-55.

7. Gill, S S & Tuteja, N 2010, ‘Polyamines and abiotic stress tolerance in plants’, Plant signaling & behavior, vol. 5, no. 1, pp. 26-33.

8. Hummel, I, Couée, I, El Amrani, A, Martin-Tanguy, J & Hennion, F 2002, ‘Involvement of polyamines in root development at low temperature in the subantarctic cruciferous species Pringlea antiscorbutica’, Journal of Experimental Botany, vol. 53, no. 373, pp. 1463-73.

9. Imai, A, Matsuyama, T, Hanzawa, Y, Akiyama, T, Tamaoki, M, Saji, H, Shirano, Y, Kato, T, Hayashi, H & Shibata, D 2004, ‘Spermidine synthase genes are essential for survival of Arabidopsis’, Plant physiology, vol. 135, no. 3, pp. 1565-73.

10. Li, H 2018, ‘Minimap2: pairwise alignment for nucleotide sequences’, Bioinformatics, pp. bty191-bty.

11. Löytynoja, A 2014, ‘Phylogeny-aware alignment with PRANK’, in D J Russell (ed.), Multiple Sequence Alignment Methods, Humana Press, Totowa, NJ, pp. 155-70, DOI 10.1007/978-1-62703-646-7_10, <https://doi.orq/10.1007/978-1-62703-646-7 10>.

12. Page, A J, Cummins, C A, Hunt, M, Wong, V K, Reuter, S, Holden, M T, Fookes, M, Falush, D, Keane, J A & Parkhill, J 2015, ‘Roary: rapid large-scale prokaryote pan genome analysis’, Bioinformatics, vol. 31, no. 22, pp. 3691-3.

13. Price, M N, Dehal, P S & Arkin, AP 2010, ‘FastTree 2-approximately maximum-likelihood trees for large alignments’, PLoS One, vol. 5, no. 3, p. e9490.

14. Richter, M & Rosselló-Móra, R 2009, ‘Shifting the genomic gold standard for the prokaryotic species definition’, Proceedings of the National Academy of Sciences, vol. 106, no. 45, pp. 19126-31.

15. Seemann, T 2014, ‘Prokka: rapid prokaryotic genome annotation’, Bioinformatics, vol. 30, no. 14, pp. 2068-9.

16. Vaser, R, Sovic, I, Nagarajan, N & Sikic, M 2017, ‘Fast and accurate de novo genome assembly from long uncorrected reads’, Genome Res, vol. 27, no. 5, pp. 737-46.

17. Walker, B J, Abeel, T, Shea, T, Priest, M, Abouelliel, A, Sakthikumar, S, Cuomo, C A, Zeng, Q, Wortman, J, Young, S K & Earl, AM 2014, ‘Pilon: an integrated tool for comprehensive microbial variant detection and genome assembly improvement’, PLoS One, vol. 9, no. 11, p. e112963.

18. Weber, T, Blin, K, Duddela, S, Krug, D, Kim, H U, Bruccoleri, R, Lee, S Y, Fischbach, M A, Muller, R, Wohlleben, W, Breitling, R, Takano, E & Medema, M H 2015, ‘antiSMASH 3.0-a comprehensive resource for the genome mining of biosynthetic gene clusters’, Nucleic Acids Res, vol. 43, no. W1, pp. W237-43.

19. Wick, R R, Judd, L M, Gorrie, C L & Holt, K E 2017, ‘Unicycler: Resolving bacterial genome assemblies from short and long sequencing reads’, PLOS Computational Biology, vol. 13, no. 6, p. e1005595.

20. Wick, R R, Schultz, M B, Zobel, J & Holt, K E 2015, ‘Bandage: interactive visualization of de novo genome assemblies’, Bioinformatics, vol. 31, no. 20, pp. 3350-2.

21. Wolf, A, Fritze, A, Hagemann, M & Berg, G 2002, ‘Stenotrophomonas rhizophila sp. nov., a novel plant-associated bacterium with antifungal properties’, International Journal of Systematic and Evolutionary Microbiology, vol. 52, no. 6, pp. 1937-44.

22. Xie, S-S, Wu, H-J, Zang, H-Y, Wu, L-M, Zhu, Q-Q & Gao, X-W 2014, ‘Plant growth promotion by spermidine-producing Bacillus subtilis OKB105’, Molecular plant-microbe interactions, vol. 27, no. 7, pp. 655-63.

1-34. (canceled)

35. A substantially purified or isolated endophyte strain isolated from a plant of the Poaceae family, wherein said endophyte is a strain of Stenotrophomonas rhizophila which provides bioprotection and/or biofertilizer phenotypes to plants into which it is inoculated.

36. The endophyte according to claim 35, wherein the bioprotection and/or biofertilizer phenotype includes production of a bioprotectant compound in the plant into which the endophyte is inoculated.

37. The endophyte according to claim 36, wherein the bioprotectant compound is spermidine, or a derivative, isomer and/or salt thereof.

38. The endophyte according to claim 35, wherein the bioprotection and/or biofertilizer phenotype is selected from the group consisting of production of organic acids, solubilization of phosphate and nitrogen fixation in the plant into which the endophyte is inoculated.

39. The endophyte according to claim 35, wherein the endophyte is Stenotrophomonas rhizophila strain JB as deposited with The National Measurement Institute on 17^{th} May 2019 with accession number V19/009906.

40. The endophyte according to claim 35, wherein the plant from which the endophyte is isolated is a pasture grass.

41. The endophyte according to claim 40, wherein the pasture grass is from the genus Lolium or Festuca, preferably from the species Lolium perenne or Festuca arundinaceum.

42. The endophyte according to claim 35, wherein the plant into which the endophyte is inoculated includes an endophyte-free host plant or part thereof stably infected with said endophyte.

43. The endophyte according to claim 35, wherein the plant into which the endophyte is inoculated is an agricultural plant selected from one or more of forage grass, turf grass, bioenergy grass, grain crop and industrial crop.

44. The endophyte according claim 43, wherein the plant into which the endophyte is inoculated is a forage, turf or bioenergy grass selected from the group consisting of those belonging to the genera Lolium and Festuca, including L. perenne (perennial ryegrass), L. arundinaceum (tall fescue) and L. multiflorum (Italian ryegrass), and those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens] x Brachiaria brizantha, [Brachiaria ruziziensis x Brachiaria brizantha] x Brachiaria decumbens; or
wherein the plant into which the endophyte is inoculated is a grain crop or industrial crop selected from the group consisting of those belonging to the genus Triticum, including T. aestivum (wheat), those belonging to the genus Hordeum, including H. vulgare (barley), those belonging to the genus Avena, including A. sativa (oats), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), those belonging to the genera Miscanthus, Paspalum, Pennisetum, Poa, Eragrostis and Agrostis; or
wherein the plant into which the endophyte is inoculated is a grain crop or industrial crop selected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, and cotton.

45. A plant or part thereof infected with one or more endophytes according to claim 35.

46. A bioprotectant compound produced by the endophyte according to claim 35, or a derivative, isomer and/or a salt thereof, preferably wherein the bioprotectant compound is spermidine or derivative, isomer and/or salt thereof.

47. A method for producing a bioprotectant compound, or a derivative, isomer and/or a salt thereof, said method including infecting a plant with the endophyte according to claim 35 and cultivating the plant under conditions suitable to produce the bioprotectant compound; or
said method including culturing the endophyte according to claim 35 under conditions suitable to produce the bioprotectant compound; and
optionally isolating the bioprotectant compound from the plant or culture medium.

48. The method according to claim 47, wherein the conditions include a culture medium including a source of carbohydrates, preferably wherein the source of carbohydrates is selected from one or more of the group consisting of a starch/sugar-based agar or broth, a cereal-based agar or broth, endophyte agar, Murashige and Skoog with 20% sucrose, half V8 juice/half PDA, water agar and yeast malt extract agar.

49. A method of providing bioprotection to a plant against bacterial and/or fungal pathogens and/or providing biofertilizer to a plant, said method including infecting the plant with the endophyte according to claim 35 and cultivating the plant.

50. The method according to claim 49, wherein the method includes providing bioprotection to the plant and includes production of a bioprotectant compound in the plant into which the endophyte is inoculated, preferably wherein the bioprotectant compound is spermidine or a derivative, isomer and/or salt thereof; or
wherein the method includes providing biofertilizer to the plant and includes production of organic acids, increased phosphate use efficiency, increased solubilisation of phosphate, increased nitrogen use efficiency and/or increased nitrogen availability, in the plant into which the endophyte is inoculated; or
wherein the method includes increasing phosphate use efficiency or increasing phosphate solubilisation in the plant, and wherein the plant is cultivated in the presence of soil phosphate and/or applied phosphate, preferably wherein the applied phosphate includes phosphate applied by fertiliser; or
wherein the method includes increasing nitrogen use efficiency or nitrogen availability, and wherein the plant is cultivated in a low nitrogen medium, preferably low nitrogen soil.

51. A method of increasing phosphate use efficiency or increasing phosphate solubilisation by a plant, said method including infecting the plant with the endophyte according to claim 35, and cultivating the plant.

52. The method according to claim 51, wherein the plant is cultivated in the presence of soil phosphate and/or applied phosphate, preferably in the presence of applied phosphate wherein the applied phosphate includes phosphate applied by fertiliser, and preferably wherein the plant is cultivated in soil.

53. A method of growing a plant in a low nitrogen medium, said method including infecting the plant with a bioprotectant compound -producing endophyte according to claim 35, and cultivating the plant.

54. The method according to claim 53, wherein the plant is cultivated in soil.


SEQUENCE LISTING (3 sequences)

SEQ ID NO: 1 - DNA, 1546 bp, Stenotrophomonas rhizophila
aaaggaggtgatccagccgcaccttccgatacggctaccttgttacgacttcaccccagtcatcggccacaccgtggcaagcgccctcccgaaggttaagctacctgcttctggtgcaacaaactcccatggtgtgacgggcggtgtgtacaaggcccgggaacgtattcaccgcagcaatgctgatctgcgattactagcgattccgacttcatggagtcgagttgcagactccaatccggactgagatagggtttctgggattggcttgccctcgcgggtttgcagccctctgtccctaccattgtagtacgtgtgtagccctggtcgtaagggccatgatgacttgacgtcatccccaccttcctccggtttgtcaccggcggtctccttagagttcccaccattacgtgctggcaactaaggacaagggttgcgctcgttgcgggacttaacccaacatctcacgacacgagctgacgacagccatgcagcacctgtgttcgagttcccgaaggcaccaatccatctctggaaagttctcgacatgtcaagaccaggtaaggttcttcgcgttgcatcgaattaaaccacatactccaccgcttgtgcgggcccccgtcaattcctttgagtttcagtcttgcgaccgtactccccaggcggcgaacttaacgcgttagcttcgatactgcgtgccaaattgcacccaacatccagttcgcatcgtttagggcgtggactaccagggtatctaatcctgtttgctccccacgctttcgtgcctcagtgtcagtgttggtccaggtagctgccttcgccatggatgttcctcccgatctctacgcatttcactgctacaccgggaattccactaccctctaccacactctagtcgtccagtatccactgcaattcccaggttgagcccagggctttcacaacagacttaaacaaccacctacgcacgctttacgcccagtaattccgagtaacgcttgcacccttcgtattaccgcggctgctggcacgaagttagccggtgcttattctttgggtaccgtcagaacaaccgagtattaatcgactgcttttctttcccaacaaaagggctttacaacccgaaggccttcttcacccacgcggtatggctggatcaggcttgcgcccattgtccaatattccccactgctgcctcccgtaggagtctggaccgtgtctcagttccagtgtggctgatcatcctctcagaccagctacggatcgtcgccttggtgggcctttaccccgccaactagctaatccgacatcggctcatctatccgcgcaaggcccgaaggtcccctgctttcacccgaaggtcgtatgcggtattagcgtaagtttccctacgttatcccccacgaaaaggtagattccgatgtattcctcacccgtccgccactcgccacccataagagcaagctcttactgtgctgccgttcgacttgcatgtgttaggcctaccgccagcgttcactctgagccaggatcaaactcttcactt

SEQ ID NO: 2 - DNA, 20 bp, Artificial Sequence
  Primer
agagtttgatcmtggctcag

SEQ ID NO: 3 - DNA, 19 bp, Artificial Sequence
  Primer
ggttaccttgttacgactt